(19) United States
(12) Patent Application Publication
(10) Pub. No.: US 2007/0275086 A1
(43) Pub. Date: Nov. 29, 2007

(54) Use of Increased Molecular-Weight Hirudin as an Anticoagulant in Extracorporeal Kidney Replace Therapy

(75) Inventors: Gotz Nowak, Erfurt (DE); Elke Bucha, Erfurt (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Max-Planck-Gesellschaft Zur Forderung Der Wissenschaften E.V

(21) Appl. No.: 11/782,365

(22) Filed: Jul. 24, 2007

Related U.S. Application Data
(63) Continuation of application No. 10/850,701, filed on May 21, 2004.
Application No. 10/850,701 is a continuation of application No. 09/585,525, filed on Jun. 1, 2000, now Pat. No. 6,423,370.

(30) Foreign Application Priority Data
Mar. 20, 2000 (EP) PCT/EP00/02446
Apr. 8, 1999 (DE) DE 19915862.2

(51) Int. Cl. A61K 35/62; A61P 13/12

(52) U.S. Cl. 424/537

(57) ABSTRACT

The invention relates to the use of increased-molecular-weight hirudin for the manufacture of an anticoagulant for extracorporeal renal replacement therapy which does not induce an autoimmune disease and which does not cross react with autoimmune antibodies. In particular, the use according to the invention does not induce type II thrombocytopenia (HIT II), and no cross reactivity occurs with antibodies to platelet-factor-4-heparin complexes.

The invention relates to the use of increased-molecular-weight hirudin for the manufacture of an anticoagulant for extracorporeal renal replacement therapy which does not induce an autoimmune disease. In particular, the invention relates to the use of increased-molecular-weight hirudin for the manufacture of an anticoagulant for extracorporeal renal replacement therapy which does not induce type II thrombocytopenia (HIT II) associated with the previously used heparins.

Extracorporeal renal replacement therapy was introduced more than 30 years ago and represents a safe method for replacing the elimination function of the kidney in cases of chronic renal failure and/or in anephric patients, using special modules which clean foreign matter from the blood using a counter-flow process. The relatively large surface areas of the capillary dialysers used in this procedure (0.5 to 2 m²) and the associated transfer of fluid and protein have a high thrombogenic potency, making the use of coagulation-inhibiting medicines an urgent necessity during haemodialysis. Heparin and/or heparin analogues are used almost exclusively in routine clinical practice. The use of heparin in patients with renal function disorders or in anephric patients has shown a whole series of side effects (osteoporosis, altered blood-lipid composition and many others), which have had to be tolerated by patients in the absence of any efficient alternatives. In recent years, a further serious side effect of heparin has become apparent: heparin-induced thrombocytopenia type II. Heparin-induced thrombocytopenia type II occurs, with a greater or lesser degree of clinical relevance, in 0.5 to 10% of all cases treated with heparin. It is an iatrogenic secondary disease with a serious thrombogenic tendency.

In this context, patients develop an antibody to the heparin-neutralising principle, platelet factor 4, in complex with heparin. This antibody only recognises the complexed platelet-factor-4-heparin structure. The antibody is not reactive or cross-reactive with heparin alone or with platelet factor 4 alone. Conversely, all heparin analogues which can form complexes with platelet factor 4 show a cross reaction. These substances also include, alongside the fractionated-heparins, polysulfated drugs of the Orgaran type or also sulfated polysugar structures which can be obtained from plant sources. The course of this immune disease is initially without symptoms in haemodialysis patients for some time, because the activated blood platelets form large-molecule aggregates with the antibody, the heparin injected into the blood and platelet factor 4, and these aggregates are held back in the capillary dialysers. At the end of the dialysis treatment, the patient is not yet heparin free, and the above-mentioned complex formation may also continue between dialysis sessions. This leads to more or less recognisable or pathogenic changes in the microcirculation of organs or vascular regions and is presumably the essential cause for haemodialysis patients becoming ill or dying from cardiovascular diseases or other organ failures during the course of renal replacement therapy much more frequently than other patients.

Without appropriate treatment, mortality after the occurrence of the first clinical symptoms is approximately 30%. This side effect demands a strict avoidance of further applications of heparin. So far, the only substitute anticoagulant agent used has been Orgaran, a mixed product containing dermatan sulfate, heparin sulfate and 5 to 10% heparin. Very many patients show primary cross-reactivity with this drug or exhibit allergic reactions after prolonged treatment with the substance. The same cross reaction is also found with low-molecular-weight heparins. Since May 1997, patients without restricted renal function have been successfully treated with the new, direct antithrombin hirudin.

There has been no shortage of experiments preparing the way for the use of hirudin for the indication of renal replacement therapy. However, since hirudin is eliminated exclusively in unchanged form via the kidneys, the dosage of hirudin must accordingly be adjusted with great caution in the case of patients with kidney disease. The lack of drug monitoring (which was introduced in clinical practice only during the last 1 to 2 years) has meant that hirudin has remained practically irrelevant for the indication of haemodialysis. Repeated application became possible only after the introduction of continuous monitoring of blood levels before and after dialysis to determine the minimum effective hirudin blood level (Ecarin Clotting Time, U.S. Pat. No. 5,547,850 dated Aug. 20, 1996, PCT/EP93/00161). Initial experience has been gathered with final hirudin anticoagulation in patients with dialysis-related HIT II (Nowak, G., Bucha, E., Brauns, I., Czerwinski, R.: Anticoagulation with r-Hirudin in Regular Haemodialysis with Heparin-Induced Thrombocytopenia (HIT II), (Wien. Klin. Wochenschr. 109, No. 10, 1997), similarly in the case of clinical studies after a single or repeated application of r-Hirudin.

In pre-clinical and initial clinical investigations, rHirudin could be used for haemodialysis only when low-flux polysulfone or low-flux cellulose dialysers were used because rHirudin does not pass through these dialysis membranes. All other low-flux dialyser materials and also all high-flux materials are passed relatively quickly by rHirudin so that universal use as an anti-coagulant in day-to-day haemodialysis practice is rendered more difficult, because under these conditions, a routine blood-level-controlled dosage adjustment is not possible (cf. Bucha, E., Kreml, R., Nowak, G.: In Vitro Study of Transmembrane Hirudin Passage Using Different Types of Dialyzers, Poster, 23 Congress of the European Renal Association, Amsterdam 1996; Buchs, E., Nowak, G., Butti, A.: Clinical Studies of Blood-Level Controlled Repeated Application of rHirudin in Haemodialysis Patients, Thromb. Haemost., Supplement June 1997; and Nowak, G., Bucha, E.: Quantitative Determination of Hirudin in Blood and Body Fluids, Sem. Throm Haemost. 22, no. 2, 1996).

The use of hirudin in special high-flux dialysers, which are used with intensive-therapy patients, is also rendered particularly difficult because these so-called haemofiltration or haemodiafiltration systems have extremely large pores through which toxins and peptides up to a molecular mass of 45 kDa can penetrate. rHirudin passes these membranes as readily as the conventional high-flux membranes and can be determined on the dialysate side within a short time. When using rHirudin, extremely large quantities would have to be infused into patients. This hirudin therapy is difficult and expensive to control, so that routine use for this indication is not possible.

There is therefore a need for an anticoagulant which can be used for coagulation inhibition during extracorporeal renal replacement therapy (haemodialysis) independently of the haemodialysers or haemofilters in use and which is well tolerated and, especially, which neither induces autoimmune diseases nor provides cross reactivity with antibodies formed during an existing immune reaction. In the case of the use according to the invention, it is particularly important that no thrombocytopenia Type II is caused and no cross reactivity exists with antibodies to platelet-factor-4-heparin complex. It should be possible to implement the coagulation inhibition simply, cost-favourably and under readily measurable conditions. Moreover, the anticoagulant should not be able to pass through the membranes of haemodialysers or haemofilters.

Surprisingly, it was found that, by contrast with rHirudin, increased-molecular-weight hirudin does not pass through capillary dialysers of the low and high flux type; it is well tolerated by patients, causes no autoimmune diseases, especially not thrombocytopenia and does not cross react with corresponding antibodies. Increased-molecular-weight hirudin is therefore very well suited as an anticoagulant for haemodialysis.

The invention therefore relates to the use of increased-molecular-weight hirudin for the manufacture of an anticoagulant for extracorporeal renal replacement therapy which does not induce an autoimmune disease. In particular, the invention relates to the use of increased-molecular-weight hirudin as defined above for the manufacture of an anticoagulant for extracorporeal renal replacement therapy which does not induce thrombocytopenia and which does not cross react with autoimmune antibodies.

Any increased-molecular-weight hirudins may be used. However, the increase in molecular weight must be such that these hirudins do not enter into an interaction with the vascular wall, with cells in the human organism and/or with blood cells, so that uncontrolled quantities are not lost.

The molecule used for increasing the molecular weight must be chemically inert and neutral. In this context, the hirudin can be coupled to any large-molecule substances using per se familiar methods. Hirudins of this type are commercially available and can be manufactured using familiar methods. These high-molecular weight compounds may be of a natural or synthetic nature. All compounds may be used which are physiologically safe and which can be coupled to hirudin. Examples of these are polyethylene-glycol-coupled hirudin, protein-coupled hirudin, DNA-RNA-coupled hirudin, polysugar-coupled hirudin, e.g. dextran hirudin, albumin hirudin, γ-globulin hirudin, transferrin hirudin, fatty-acid-coupled hirudin, (e.g. palmitoyl hirudin, farnesyl hirudin) and others. By preference, polyethylene-glycol-coupled hirudin is used.

The molecular weight of the hirudin is generally increased with substances such that the resulting increased-molecular-weight hirudins show no tendency to bind to proteins, blood platelets and other blood constituents. The increase in molecular weight is selected by the person skilled in the art in dependence upon the condition to be treated and the mode of administration. In general, it is within the range between 500 and 500,000 Da, preferably 1,000 to 250,000 Da, by greater preference 3,000 to 150,000 Da and by even greater preference 5,000 to 25,000 Da. The increased-molecular-weight hirudins are manufactured in a per se familiar manner using conventional formulation additives in accordance with the relevant mode of administration.

The hirudin blood level required for haemodialysis should preferably be adjusted between 0.4 and 1.0 μg/ml plasma. Within this blood-level range, activation of the coagulation system on the polymer surfaces of the dialysis modules used in this context is efficiently inhibited. However, the hirudin blood level to be adjusted depends on the physiological condition of the patient to be treated and will be adjusted by a physician in dependence upon patient-typical factors.

When using increased-molecular-weight hirudin, the blood level should preferably be achieved by means of intravenous bolus injection of e.g. 0.01 to 0.1 mg/kg polyethylene-glycol hirudin (for discontinuous extracorporeal renal replacement therapy). For use in the field of intensive therapy for haemofiltration/haemodiafiltration, the application of PEG-hirudin is also possible via extravasal application, such as intramuscular, intraperitoneal, pulmonary or subcutaneous routes. In this context, the application of e.g. polyethylene-glycol hirudin should be given subcutaneously at least two hours before the start of haemofiltration, in a dose of 0.2 to 1.5 mg/kg, preferably 0.5 to 0.7 mg/kg. Further subcutaneous injections would be required only after 48 hours in each case (0.1 to 0.5 mg/kg, preferably 0.2 to 0.3 mg/kg PEG-hirudin). These dosage amounts are calculated with reference to the active hirudin, independently of the inert substance used for increasing the molecular-weight. In the case of protein-coupled hirudins (e.g. transferrin, albumin or other autogenous, blood proteins) and PEG hirudins with a molecular weight >50 kDa, the following dosage scheme has proved successful: initial dose of hirudin: 0.05 mg/kg as a bolus; for each further injection 0.01 mg/kg as a bolus directly before the start of the dialysis. In the case of continuous procedures (haemodialysis or haemofiltration) the initial application of 0.05 mg/kg, with 0.01 mg/kg after 48 hours as a follow-up injection in each case every second day during the changing of the haemofilter as a single intravasal application is sufficient. Because of the size of the molecules, parenteral injection procedures (s.c., i.m.) are not suitable for this special group of increased-molecular-weight hirudins. All increased-molecular-weight hirudins from 500 Da to 500 kDa can be applied directly into the patient's blood stream (i.v. or i.m.). Up to a molecular size of approximately 50 kDa, the application may also be extravasal or parenteral, i.e. intramuscular, subcutaneous, intracutaneous or intrapulmonary. This is not possible above the latter threshold molecule size, because these substances with a large molecular size cannot be absorbed from the extravasal injection depots into the interior of the blood vessels. Molecules of this size cannot pass through intercellular spaces. If very large molecules are consciously used as hirudin carriers, especially proteins of nucleic-acid macromolecules and other appropriate molecules larger than 50 kDa, it can be assumed that these molecules will not leave the vascular circulation, i.e. they will be distributed only within the blood (stream). The ratio between the blood (stream) and the extracellular fluid cavity, which represent the two distribution parameters for molecules smaller than 50 kDa, is approximately 1:5, i.e. with the very large hirudin molecules, a significantly smaller quantity needs to be applied intravenously or intrarterially if the development of an excessively high blood level is to be avoided.

The exact dosage depends on the increased-molecular-weight hirudin used, the patient's condition, the mode of administration and the duration of treatment, and this is determined individually by the treating physician. The frequency of administration is also specified by the physician. The therapeutic blood level can be monitored without difficulty using the Ecarin Clotting Time for therapeutic drug monitoring (Method of Determining Hirudin, U.S. Pat. No. 5,547,850, PCT/EP93/00161). Overdosage of the PEG-hirudin substance is precluded if the pharmacokinetic data for PEG-hirudin are taken into consideration (Esslinger, H. U. et al.: Pharmacodynamic and Safety Results of PEG-Hirudin in Healthy Volunteers, Thromb. Haemost. 77 (5), 1997). Should an iatrogenic overdose occur, the use of a polymethyl methacrylate dialyser can lower the level of PEG-hirudin very quickly into the therapeutic range (PMMA membranes with polyethylene-glycol-coupled active substances, DPA 197 15 504.9 of Apr. 14, 1997). This therefore makes an “antidote” available for PEG-hirudin anticoagulation in cases of renal replacement procedures, because PMMA-dialysers are produced commercially by Toray, Japan.

The increased-molecular-weight hirudins according to the invention are formulated in a per se familiar manner depending on the mode of administration. Conventional formulation additives, excipients and corrective agents are used. By preference, substances which improve the solubility and stability of dry preparations are used, such as, sugar compounds (mannitol, dextran), inert proteins (albumin) or collagens (Prionex). Moreover, to improve storage, salts, primarily buffer salts, such as e.g. bicarbonates or hydrogen phosphates and other salt compounds conventional in the field of formulating buffer systems, may be added. By preference, ampoules are manufactured, which provide ready-to-use injection solutions taking into consideration the weight-related dosage, e.g. 1, 2 or 5 mg ampoules. It is expedient to add these substances, added in order to improve the solution or as formulation additives, in a molecular ratio of 1:1. Additions in the range between one fifth and 1:1 relative to the molecular weight of the increased-molecular-weight hirudin substances have also proved favourable. The solution should be formulated in such a manner that the ready-to-use increased-molecular-weight hirudin comprises a concentration which is isotonic to the blood. During formulation, it is important to ensure that buffer salts or saline are added within the range of optimum blood isotonicity in order to guarantee the safety of the preparations when applied into the blood stream. The increased-molecular-weight hirudin can also be provided in freeze-dried form in air-tight ampoules or injection vials, which can then be diluted to the required concentration, for instance, with physiological saline solution of pharmaceutical quality.

The use according to the invention is suitable for all commercially available haemodialysers.

The invention will be explained in greater detail below with reference to the following examples:

### EXAMPLE 1

### Scheme For Application of PEG-Hirudin (10-kDa-PEG-Hirudin) For Anticoagulation With Discontinuous Haemodialysis Procedures Using All High-Flux Or Low-Flux Dialysers (With the Exception of PMMA Dialysers)

For the first haemodialysis, a bolus injection of 0.08 mg/kg 10-kDa-PEG-hirudin is administered intravenously directly before haemodialysis treatment.

For subsequent haemodialysis treatments an i.v. bolus injection of 0.06 mg/kg 10-kDa-PEG-hirudin is administered.

Monitoring of the PEG-hirudin level with reference to the Ecarin Clotting Time at the end of the haemodialysis treatment. The therapeutic blood-level range is 0.5 to 0.8 μg/ml.

### EXAMPLE 2

### Scheme For I.V. Application of PEG-Hirudin (10-kDa-PEG-Hirudin) With Continuous Dialysis Procedures (CVVHD, CAVHD)

Directly before the start of the continuous haemodialysis, 0.1 mg/kg 10-kDa-PEG-hirudin is administered i.v. at a position preceding the dialyser used.

After 24 and/or 48 hours, the PEG-hirudin blood levels are monitored with reference to the Ecarin Clotting Time.

After 24 hours, an optimum blood-level range of 0.8 to 0.5 μg/ml is obtained; after 48 hours, an optimum blood-level range of 0.4 to 0.6 μg/ml is obtained.

After the change of dialyser, a follow-up application of 0.02 mg/kg 10-kDa-PEG-hirudin is administered.

### EXAMPLE 3

### Scheme For S.C. Application of PEG-Hirudin (10-kDa-PEG-Hirudin) With Continuous Dialysis Procedures

(a) In patients with renal function still present:
2 hours before the start of the haemodialysis procedure, a subcutaneous application of 0.25 to 1.0 mg/kg, preferably 0.6 mg/kg PEG-hirudin is administered.
The blood level is monitored 24 hours and 48 hours after the start of the treatment.
Every 2 to 4 hours, a follow-up injection of 0.1 to 1 mg/kg, preferably 0.3 mg/kg PEG-hirudin is administered.

(b) In patients with renal function absent:
At the start of the treatment, a s.c. application of 0.2 to 1.0 mg/kg, preferably 0.4 mg/kg.
The blood level is monitored p.i. 24 hours and 48 hours after the start of the treatment.
Every 4 days, a follow-up injection of 0.01 to 0.25 mg/kg, preferably 0.1 mg/kg PEG-hirudin is administered.

### EXAMPLE 4

### Application of PEG-Hirudin In Status Post Chronic Glomerulonephritis

Patient: W. B., 58 years, male. Status post chronic glomerulonephritis, dialysis-dependent for 4.7 years. Patient was anticoagulated with 6900 IU heparin. During haemodialysis, the patient indicated increasing symptoms in the form of circulatory disturbances in the extremities, sensations of cold, tingling sensations, paraesthesias, which frequently did not arise until several hours after the dialysis treatment or in the night and early hours of the morning. The patient complained during dialysis and also in the dialysis-free intervals of problems relating to blood pressure and in some cases mild states of confusion. A positive result had been obtained in the HIT II-specific diagnosis (HIPA-test). The average number of blood platelets before and after haemodialysis was between 350 and 400/nl.

The patient was transferred to PEG-hirudin according to the following dosage scheme: first haemodialysis: 0.1 mg/kg as a bolus, second to 10^{th} haemodialysis: 0.05 mg/kg PEG hirudin, 11^{th} haemodialysis and beyond: 0.025 mg/kg. Trouble-free monitoring was possible during the treatment with reference to the Ecarin Clotting Time. The blood level of PEG hirudin fluctuated between 0.4 μg/ml before the start of haemodialysis and 1.0 μg/ml after dialysis. The filling volumes with the Type GFS plus 11 dialysers (GAMBRO) were as follows: under heparin application—the mean value for the last three heparin-anticoagulated dialyses was 87 ml; the filling volume during PEG-hirudin treatment for HD 5-HD 6 was 96 ml. The filling volume specified for this type of dialyser is 100 ml.

The patient was suffering from an arterial circulatory disturbance in the extremities. During treatment with heparin-anticoagulated haemodialysis, the patient's pain-free walking distance was 150 to 200 m; under PEG hirudin treatment, this distance was extended, so that during monitoring after the tenth haemodialysis, it was more than 700 m. As treatment continued, the patient indicated a further improvement in his pain-free walking distance. Even 8 weeks after starting the PEG-hirudin treatment because of an HIT II, HIT-II-specific antibodies were detected in the patients serum.

Even after the initial PEG-hirudin dialyses, the patient's symptoms of paraesthesia and nocturnal pain attacks were reduced. After the 7^{th} PEG-hirudin haemodialysis, the patient confirmed that he was free from pain during haemodialysis and in the dialysis-free intervals.

Evaluation: PEG-hirudin is suitable as an anticoagulant for haemodialysis in HIT II-positive patients. The symptoms of chronic microcirculatory disturbance with a permanent HIT II reaction during heparin treatment of the patient in the context of haemodialysis did not occur under PEG-hirudin. The short, pain-free walking distance occurring on the basis of an intermittent claudication was considerably extended, and the circulatory dysregulation and considerably reduced filling volume of the dialysers was no longer present.

### EXAMPLE 5

### Application of PEG-Hirudin In A Case of Diabetic Nephropathy

Patient: G. W., male, 57 years old, 54 kg. The patient has been haemodialysed for 2.4 years because of a diabetic nephropathy. The patient had a residual urine volume of 1.2 to 1.7 l. Creatinine clearance was less than 12 ml/min. The anticoagulant used during haemodialysis was un-fractionated heparin. Additionally, the dialysers (H120, Braun-Melsungen) were “primed” before the start of haemodialysis with 500 units of heparin. The patient received 3000 units of heparin as a bolus and 3000 units of heparin as an infusion during the 3.5 hour haemodialysis. At the start of the haemodialysis, the patient showed a disproportionately large fall in blood pressure by 35 mmHg from the starting value (135/85 mmHg). The patient complained of tingling in the extremities, a feeling of restlessness and intermittent state of confusion. Between the dialysis sessions, the patient complained of headaches, malaise and loss of appetite. He stated that the people around him complained that he was irritable and seemed under stress. Tests for HIT-positive antibodies in this patient were strongly positive. Both the HIPA and the ELISA tested positive in all experimental series investigated.

Following this, the patient was transferred to PEG-hirudin. He received 0.1 mg/kg PEG hirudin as the first dose. In subsequent haemodialyses, he was treated with 0.06 mg/kg up to the fifth haemodialysis and with 0.03 mg/kg from the sixth treatment onwards. The filling volumes at the end of haemodialysis had been reduced under heparin treatment; the last two haemodialyses under heparin showed filling volumes of 71 and 73 ml. Even after the 4^{th} haemodialysis with PEG-hirudin, a filling volume of 94 ml was achieved. The air traps and blood-carrying tubing systems were free from additional coagulated material.

The patient's subjective condition had improved; the psycho-organic syndrome and the patient's other recorded symptoms such as headaches, nausea and loss of appetite were no longer present. The patient still showed a slight fall in blood pressure at the start of the haemodialysis, but this was within the normal range. The objective and subjective findings which point towards an HIT II condition were no longer evident after the changeover to anticoagulation with PEG-hirudin. The blood platelet counts, which had constantly shown values above 400/nl during the last dialyses under heparin, fell during the PEG-hirudin haemodialysis to the range from 320 to 350/nl; a trend towards continued normalisation was clearly demonstrable. The post dialysis values for leukocytes were almost unchanged.

### EXAMPLE 6

### Application of PEG-Hirudin In A Case of Acute Renal Failure

Patient: L. A., female, 69 years, status post acute renal failure after chronic pyelonephritis and interstitial nephritis. This patient had been dialysed for 5 months initially with un-fractionated heparin, then with fractionated heparin (Enoxaparin). Enoxaparin was applied in a dose of 20 mg directly before the start of the dialysis. After an incorrect dialysis (rise in pressure before the dialyser) over the dialysis period of 4.5 h, an additional 10 mg Enoxaparin were applied via an indwelling infusion pump placed before the dialyser (F 50 S, Fresenius). The patient indicated severe shortness of breath, a decline in circulation and blood pressure to base values of 80/30 mmHg shortly after the start of haemodialysis. During the haemodialysis, the patient also showed a massive reduction in blood platelets down to values of 100/nl. Tests for HIT II antibodies using the HIPA test and platelet-factor-4 ELISA were positive.

The patient was then transferred to PEG-hirudin. Residual urine volume: 400 to 600 ml/24 h, initial PEG-hirudin dose: 0.1 mg/kg, second to 10^{th} haemodialysis: 0.05 mg/kg PEG-hirudin, 11^{th} and further haemodialysis: 0.02 mg/kg. From the start of the application of PEG-hirudin, the patient's circulatory changes and severe pulmonary insufficiency reaction no longer occurred; the concentration of blood platelets did not decline during haemodialysis. The reduced platelet counts (130 platelets/nl) caused by the relative thrombocytopenia present in this patient rose within the first 6 haemodialyses with PEG-hirudin to more than 200/nl. The itching and skin-reaction syndrome which had been present for several weeks subsided within a few haemodialyses. The patient was free from symptoms and 6 months after the start of the PEG-hirudin dialysis showed no further HIT II antibodies in her blood. The patient's PEG-hirudin blood level, adjusted by means of the drug-monitoring method, was within the range of 0.5 μg/ml at the end of the haemodialysis and during the dialysis-free intervals of 1 to 2 days fell to a minimum of 0.15 μg/ml.

In summary, it can be confirmed that the base PEG-hirudin blood level demonstrable in all three patients during the dialysis-free intervals is within the range of 0.1 to 0.4 μg/ml; this level is therefore within the sub-therapeutic range and is completely free from side effects. No prolongation of bleeding time was demonstrable in any of the three patients even during dialysis treatment with blood levels two to three times higher. Permanent anticoagulation improves the holding open of the arterio-venous shunts and must be evaluated positively with regard to the overall risk of thrombophilia present in all patients.

The administration of increased-molecular-weight hirudin prevents patients treated in this manner from showing the typical autoimmune reactions. These include a fall in blood pressure, shortness of breath, sweating bouts, skin reactions in some cases, such as urticaria or circumscribed drug-related exanthema which are immediately demonstrable in the patient as an instant reaction to the triggering agent, i.e. heparin. These autoimmune reactions can, at their most severe, lead to life-threatening conditions. Typical of these reactions is a no-longer-measurable blood pressure and extremely severe thrombogenic reactions with obstruction of the capillary dialyser, to such an extent that efficient dialysis cannot be continued. The dialysers must then be changed (in some cases up to three times during a dialysis session), in order to sustain the corresponding filter functions. By contrast, these side effects no longer occur in patients treated with the increased-molecular-weight hirudin. In this case, consistently stable circulatory conditions are present and patients do not suffer from subjective sensations such as tingling flesh, flash reactions (reddening of the head and neck regions) fall in blood pressure with severe signs of breathlessness and fear of death. The use of increased-molecular-weight hirudin according to the invention is indicated primarily for patients in whom the serious symptoms described above occur as a result of heparin treatment. However, a more frequent sign for a transitory HIT II syndrome in these patients is that the blood platelet counts fall more markedly during dialysis, that corresponding antibodies can be demonstrated in these patients before the start of the dialysis, but that these antibodies are no longer found after haemodialysis because they have been “used up” in the process.

1-5. (canceled)

6. A method of treating a patient suffering from renal failure, the method comprising administering to the patient an effective amount of an increased-molecular-weight hirudin selected from polyethylene-glycol-coupled hirudin, polysugar-coupled hirudin, fatty-acid-coupled hirudin, dextran hirudin, albumin hirudin, γ-globulin hirudin and transferrin hirudin.

7. A method according to of claim 15, where the autoimmune disease is a heparin-induced thrombocytopenia Type II.

8. A method according to claim 15, where the increased-molecular-weight hirudin is administered via the vasal or extravasal route.

9. A method according to claim 7, the increased-molecular-weight hirudin is administered via a vasal or extravasal route.

10. A method according to claim 15, where the effective amount of the increased-molecular-weight hirudin is a dosage sufficient to provide a blood plasma level of hirudin in the range from 0.4 to 1.0 μg/ml plasma.

11. A method according to claim 7, where the effective amount of the increased-molecular-weight hirudin a dosage sufficient to provide a blood plasma level of hirudin in the range from 0.4 to 1.0 μg/ml plasma.

12. A method according to claim 8, where the effective amount of the increased-molecular-weight hirudin is a dosage sufficient to provide a blood plasma level of hirudin in the range from 0.4 to 1.0 μg/ml plasma.

13. A method according to claim 9, where the effective amount of the increased-molecular-weight hirudin is a dosage sufficient to provide a blood plasma level of hirudin in the range from 0.4 to 1.0 μg/ml plasma.

14. A method of treating a patient suffering from renal failure, the method comprising administering to the patient a pharmaceutical composition comprising an effective amount of an increased-molecular-weight hirudin selected from polyethylene-glycol-coupled hirudin, polysugar-coupled hirudin, fatty-acid-coupled hirudin, dextran hirudin, albumin hirudin, y-globulin hirudin and transferrin hirudin.

15. A method of treating a patient suffering from renal failure, the method comprising administering to the patient an effective amount of a hirudin conjugate which does not induce an autoimmune disease, where the hirudin conjugate is an increased-molecular-weight hirudin selected from polyethylene-glycol-coupled hirudin, polysugar-coupled hirudin, fatty-acid-coupled hirudin, dextran hirudin, albumin hirudin, γ-globulin hirudin and transferrin hirudin.

16. A method according to claim 5, where the administering is in connection with extracorporeal renal replacement therapy.

17. A method according to claim 14, where the administering is in connection with extracorporeal renal replacement therapy.

18. A method according to claim 15, where the administering is in connection with extracorporeal renal replacement therapy.
